(19) United States
(12) Patent Application Publication
(10) Pub. No.: US 2026/0227295 A1
(43) Pub. Date: Aug. 6, 2026

(54) Breath Sampling Device and Method for Preparing a Sample From a Breath Sampling Device for Analysis

(71) Applicant: Owlstone Medical Limited, Cambridge (GB)

(72) Inventors: Alastair Taylor, Cambridge (GB); David Jessop, Cambridge (GB); Desirèe Otero, Cambridge (GB); Jacob Beasley, Cambridge (GB); Jenifer Mizen, Cambridge (GB); Mohammad Alkaseem, Cambridge (GB); Nidhin Shaju, Cambridge (GB); Rob Smith, Cambridge (GB); Simon Kitchen, Cambridge (GB); Max Allsworth, Cambridge (GB)

(73) Assignee: Owlstone Medical Limited, Cambridge (GB)

(21) Appl. No.: 19/150,684

(22) PCT Filed: Feb. 2, 2024
(86) PCT No.: PCT/GB2024/050298

§ 371 Date: Jul. 24, 2025

(30) Foreign Application Priority Data
Feb. 3, 2023 (GB) 2301582.9
Feb. 3, 2023 (GB) 2301583.7

(51) Int. Cl. G01N 1/22; A61B 5/097; B01D 53/04; B01D 53/26; G01N 1/38; G01N 1/40

(52) U.S. Cl. CPC G01N 1/2214; A61B 5/097; B01D 53/0438; B01D 53/0446; B01D 53/0454; B01D 53/261; G01N 1/38; G01N 1/4022; B01D 2257/708; B01D 2257/80; B01D 2259/40088; B01D 2259/4533; B01D 2259/4541; G01N 2001/2244; G01N 2001/386

(57) ABSTRACT

The present techniques relate to breath sampling device and a method for preparing a collected sample from a breath sampling device. The breath sampling device comprises a filter (204), a drying component (210), a mixer (220) and a sampler (230). The drying component (210) is positioned in the fluid path and it is configured to split the fluid path into first and second pathways which include a drying bed (214) and one or a plurality of bypass channels (212). The drying component may comprise a sieve comprising a drying bed of drying material held between an inlet face (202) and an outlet face (206). The method comprises simultaneously releasing and drying a collected sample from the sorbent material by heating the sorbent material to release the collected sample and mixing the released collected sample with a dry gas to dry the released collected sample. The dried released collected sample is then collected ready for analysis.

### TECHNICAL FIELD

The present techniques relate to breath sampling devices, and optionally to methods and systems for preparing samples from such breath sampling devices for analysis.

### BACKGROUND

FIG. 1a shows a ReCIVA® Breath Sampler which is a non-invasive breath sampling device 10 and which is described for example in patent publications WO2017/187120 or WO2017/187141. The breath sampling device 10 can collect volatile organic compound and respiratory droplet samples from exhaled breath. The breath sampling device 10 provides non-invasive sampling during normal tidal breathing with options for breath fraction targeting while ensuring patient safety and comfort. The device 10 comprises a plurality of sorbent tubes 12 which contain a sorbent material which is used to sample the breath.

FIG. 1b shows an alternative breath sampler which is configured to sample a significant portion (perhaps the entirety) of an exhaled breath from a user/patient. This is achieved by using a sampling device comprising adsorbent material having a geometry adapted for optimal results as explained below. The optimised geometry is one in which the thickness of the material is significantly smaller than the width of the material. Such a breath sampler is described in PCT/GB2022/051927 to the present applicant. The breath sampling device 120 comprises a housing 150 and an adsorber material holder 152 within the housing 150. The adsorber material holder 152 comprises an adsorber material and extends across most of the cross-section of the housing 150. The device comprises an inlet 130 and an outlet 160 which are fluidly connected by the housing 150 and the adsorber material holder 152 is supported on the fluid path between the inlet and the outlet to sample the breath of the user/patient. The inlet 130 is configured to accommodate the mouthpiece 134. A bypass portion 140 extends from the housing 150, generally at right angles to the fluid path. A one-way valve 146 may be connected to the inflatable element to stop fluid flowing back through the bypass portion 140.

Humidity can be a problem when trying to accurately measure an amount of a volatile organic compound (VOC) within a breath sample. For example, when the VOC which is being targeted is D5 ethanol (also known as ethyl-d5 alcohol), it is known that the sorbent material's ability to adsorb the target analyte decreases as the humidity increases. This is illustrated for example in FIG. 1c. FIG. 1c shows the change in the relative amount of D5-ethanol adsorbed on the sorbent material decreases with the relative humidity of the sample. This is demonstrated for two sorbent materials, carbosieve SIII® and carboxen® 569 . FIG. 1c was generated from a fixed volume collection of D5-ethanol in 2.1 L of air and a fixed sorbent mass of 100 mg. It has also been proposed that the water is removed from the sorbent material before analysis. Typically, some of the target analyte is removed with the water. Avoiding losing the target analyte can lead to longer processing times and costs.

FIG. 1d illustrates a process which may be used to remove water which is present in the breath sample. In a first step S100 a breath sample is collected, for example using the prior devices described above. At step S102, the sorbent material (also termed sampler) is extracted. The sorbent material may be in the form of a disc or any other shaped component. The extracted sorbent material is then sent to a laboratory for analysis.

At step S104, the sorbent material is purged to remove water from the sorbent material. After purging, the breath sample is transferred at step S106 from the sorbent material to a recapture device (e.g. sorbent tube). The recapture device may be then analysed using any suitable device at step S108. It will be appreciated that some of the VOC may be lost during any or all of the following steps: when collecting the sample at step S100, when sending the breath sample (on its sampler) to a laboratory, when purging the sorbent at step S104, when transferring the sample from the sampler to a standard sorbent tube at step S106 and when analysing at step S108. The largest loss of target VOC is typically when purging water from the sorbent material which contains the breath sample.

The applicant has recognised the need for a device and/or processing system with improved humidity characteristics.

### SUMMARY

According to the present techniques, there is provided a device, a method and a system as set forth in the appended claims. Other features of the invention will be apparent from the dependent claims, and the description which follows.

There is provided a breath sampling device comprising an inlet through which a breath sample is received, an outlet through which the breath sample exits the breath sampling device and a fluid path between the inlet and outlet. Thus, in use a breath sample flows down through the device from the inlet to the outlet. The breath sampling device comprises a drying component which is positioned in the fluid path and which is configured to split the fluid path into first and second pathways (also termed channels). In other words, in use, a first portion of a breath sample passes through the first pathway and, at the same time, a second portion of the breath sample passes through the second pathway. The breath sampling device also comprises a mixer which is positioned in the fluid path downstream from the drying component and which is configured to recombine the first and second pathways into a single fluid path. In other words, in use, the mixer is configured to mix the first and second portions of the breath sample. The breath sampling device also comprises a sampler which is positioned in the fluid path downstream from the mixer and which is configured to collect a target compound from a breath sample which is received through the inlet.

The first pathway comprises drying material and may be termed a drying channel. The drying material may be any suitable material which removes water from a first portion of the breath sample as it passes through the first pathway. Thus, a dried first portion of the breath sample exits the first pathway. The second pathway is separated from the first pathway and the drying material. The second pathway may thus be termed a bypass channel through which, in use, a second portion of the breath sample simultaneously passes through the second pathway without any drying. The first portion passes through the first pathway at the same time as the second portion passes through the second pathway. The first and second pathways are combined in the mixer and thus the dried first portion of the breath sample and the second unchanged portion of the breath sample are mixed in the mixer to produce an output breath sample with lower humidity that the breath sample received in the inlet. For example, the output breath sample may have a humidity of approximately 40% and the second portion (and hence input breath sample) may have a humidity level of between approximately 60 to 80%. The dried first portion may have a humidity level of between 0 to 10%.

The drying component may comprise an inlet face and an outlet face defining a cavity therebetween. A drying bed of drying material may be held between the inlet face and the outlet face and may occupy at least a portion of the cavity. Each inlet and outlet face may comprise a mesh having a plurality of pores and the first portion of the breath sample flows through at least some of the pores in the mesh into the drying bed. In other words, the first pathway is defined as the pathway through the mesh in the inlet face into the drying bed and out through the mesh in the outlet face. The drying bed may be a sieve, more preferably a molecular sieve whereby as a mixture of molecules migrate through the drying bed, the components of highest molecular weight are unable to pass through the drying bed and leave the bed first followed by successively smaller molecules.

The drying component may further comprise at least one channel which connects the inlet face to the outlet face and which is separated from (i.e. bypasses) the drying bed. There may be a plurality of such bypass channels and each channel may be in the form of a tube. There may be a central bypass channel. Other bypass channels may be equally spaced around the inlet and the outlet faces, e.g. towards the edges of the inlet and the outlet faces or around the central channel when used. These channels may be termed outer bypass channels. The second pathway is thus defined as the passage through the or each tube and the first pathway is defined as the passage around the or each tube.

The inlet face and/or the outlet face may comprise an aperture which aligns with each bypass channel. Alternatively, the mesh in the inlet and outlet faces may cover the inlet and/or outlet for one or more bypass channels. In such an arrangement, the second pathway may flow through the mesh of the inlet face into the bypass channel and out through the mesh of the outlet face. Where separate apertures are used, each of the plurality of the apertures may be small compared to the inlet and outlet faces, e.g. less than 10% of the width/diameter. The plurality of apertures may comprise a central aperture and multiple apertures which are equally spaced around the central apertures. Alternatively, there may be no central aperture and the central bypass channel is aligned with the mesh. Each of the plurality of apertures may be larger on an upstream side (i.e. external side) of the inlet face than the downstream side (i.e. internal side connecting with the tubes) of the inlet face. In other words, each of the plurality of apertures is tapered, perhaps by approximately 20%. The tapering may apply in reverse to the outlet face. In other words, each of the plurality of apertures may be smaller on an upstream side (i.e. internal side) of the outlet face than the downstream side (i.e. external side adjacent the mixer) of the outlet face.

In another arrangement, the drying component may comprise a first chamber and a second chamber which are separated from one another. The first and second chamber may provide the cavity between the inlet and outlet faces. The first chamber comprises drying material to form the drying pathway and the second chamber provides the second pathway and may be considered a bypass channel. The two chambers may be equal in size and thus comprise two distinct halves along the axis of flow, to achieve a constant split-ratio across all flow rates. For example, a 50:50 split may be achieved. Each chamber may be filled with materials which are mechanically identical (bead size and geometry), but chemically different (one material removes water from the airstream, the other is chemically inert). The first chamber may be filled with a molecular sieve (to dry the first portion of breath) and the second chamber may be filled with chemically inert beads of the same size and geometry (through which the second portion breath may flow without any change, i.e. without being dried). When the chambers are identical in size, half of the flow may be dried and half may be undried. Each portion of breath may have the same pressure-flow characteristics across all flow rates and hence a constant split ratio.

The first and second pathways may be configured so that a ratio of a pressure drop across the second pathway and a pressure drop across the first pathway is constant. The ratio may be constant across a broad range of flowrates. In this way, in use, the same amount of breath sample is maintained through each pathway in the drying component to the mixer. More of the breath sample may pass through the first pathway. For example, the pressure ratio may vary between 1:1 so that the breath sample is divided equally between the two pathways and 6:4 so that 40% of the breath sample passes through the first pathway and 60% through the second pathway. In other words, a fixed first proportion of between 40% to 50% of the breath sample may pass through the first pathway and a corresponding fixed second proportion of between 50% to 60% of the breath sample may pass through the second pathway.

The device, in particular the first and second pathways, may also be configured so that a user can comfortably and easily breathe through the device, and the device can collect as much volatile organic compounds (VOCs) as possible. That is, the device is designed such that breath capture may be termed unassisted because the user provides the necessary pressure to push the breath through the device. In other words, the device may be optimized so that the pressure drop across one or both of the first and second pathways is not prohibitive to a user providing a breath sample. A range of ideal values for airflow which is possible through the device may be between 35 and 150 L/min and the ranges for unacceptable values for airflow are below 25 and above 200 L/min.

Configuring the first and second pathways may mean selecting the parameters (e.g. dimension (length/width)) to achieve the desired pressure drops. For example, in the arrangement described above, the parameters of the second pathway may comprise one or more of the number of the bypass channels, the length of each bypass channel and a cross-sectional area of each bypass channel (alone or in combination). It will be appreciated that for bypass channels having a circular cross-sectional areas, one of the parameters may be diameter. In other words, the second pathway may be considered to have at least one of a predetermined length, a predetermined cross-sectional area (or diameter) and/or a predetermined number of bypass channels and these predetermined parameters may be determined based on the flowrate of the breath sample through the first and second pathways. Merely as an example, the predetermined diameter of each bypass channel may be 2.05 mm; the predetermined length of each bypass channel may be 40 mm and/or the predetermined number of bypass channels may be between one and five. The central bypass channel may have a larger diameter than the outer bypass channels.

As described above, the first pathway may comprise at least one drying bed which holds the drying material. For example, the drying material may be multiple molecular sieve pellets (or beads—the terms may be used interchangeably) which remove water from the first portion of the breath sample. When configuring the first pathway, the parameters of the first pathway may comprise some or all of the mass of the drying material (i.e. the mass of the molecular sieve), the size of each molecular sieve pellets, the pore size of the molecular sieve and other aspects of the geometry of the molecular sieve. These parameters may be selected to achieve the desired level of reduction in the humidity of the first portion of the breath sample and/or selected to achieve the desired pressure drop across the first pathway. Glass beads may be included in combination with the drying material. The purpose of the glass beads is to provide a mechanical obstruction similar to the one produced by the drying material. The glass beads may not adsorb water. In this way, the fluid path may contain an optimal volume of drying material to adsorb the required amount of water while maintaining the same pressure drop.

The sampler may comprise a housing in which a sorbent material is housed. The sorbent material may be made from activated carbon and/or zeolites and/or activated alumina and/or lignite coke and/or bentonite and/or any material which is suitable to collect the target compound within the breath sample. A suitable material is, for example, carbosieve® SIII 60-80 and/or Carboxen® 569, for example when targeting D5 ethanol. The sorbent material may be shaped as a disc or any suitable shape and may extend across at least a substantial part of the cross-section of the housing of the sampler. As described above, the target humidity for the output breath sample from the mixer may be less than 40% relative humidity. The target humidity is set based on the threshold below which the sorbent material adsorbs little water. In other words, low relative humidity of the breath sample aids the absorption of volatile organic compounds (VOC) by the sorbent material. Additionally, the sorbent material adsorbing less water improves the subsequent processing and analysis. The sorbent material may adsorb other compounds as well as the target VOC and the compounds adsorbed by the sorbent material may thus be referred to as the sample.

According to another aspect, there is provided a method for preparing a collected sample from a breath sampling device for analysis, the method may comprise extracting sorbent material containing the collected sample from the breath sampling device and placing the extracted sorbent material in a chamber. The method may then comprise simultaneously releasing and drying the collected sample from the sorbent material by heating the sorbent material in the chamber to release the collected sample and mixing the released collected sample with a dry gas. The dried breath sample is then captured in a recapture device for analysis.

The method may further comprise flowing a second gas through the sorbent material while the sorbent material is being heated. This flow may facilitate releasing the sample from the sorbent material. The simultaneous releasing and drying of the collected sample may control the humidity of the sample to optimise capture in the recapture device. The method may further comprise controlling the temperature of the dried released sample to optimise capture in the recapture device. The flow rate of one or both of the dry gas and the second gas may be controlled, e.g. by using a controlled flow rate gas source.

According to another aspect, there is also provided a system for preparing a collected sample from a breath sampling device for analysis, the system comprising: a chamber which is configured to receive sorbent material containing the collected sample from the breath sampling device, a heat source for heating sorbent material received in the chamber to release the collected sample from the sorbent material; a first gas source for providing a flow of dry gas which is mixed with the released sample as the sample is released, whereby the sample is simultaneously dried and released from the sorbent material; and a recapture device which is configured to capture the dried sample to be analysed.

The following features apply to both the method and system described above.

The chamber may be filled with a gas, e.g. nitrogen, so that there is a dry atmosphere (or low humidity atmosphere) in the chamber. As explained above, the breath sample is dried by mixing the sample as it is released from the sorbent material with dry gas. A dry gas may be one with less than 10% humidity. The dry gas may be dry nitrogen. Similarly, the second gas may be dry and/or may be nitrogen. A dried sample means a sample with lower humidity than the breath sample being received by the breath sampling device, for example the dried sample may have less than 40% relative humidity. As above, there may be a controlled flow rate gas source for one or both of the dry gas and the second gas. In general, the flow of all gas sources within the system may be controlled.

The system may further comprise a humidity sensor for sensing the humidity of the dried, released sample. The system may further comprise a controller to monitor the sensed humidity and adjust the flow of at least one of the dry gas and the second gas in response to the sensed humidity. Increasing the flow rate through the disk will speed up the transfer process but the humidity must be kept below the target threshold (e.g. of 40%). For example, the flow of the dry gas may be increased if the humidity of the dried sample is above the desired threshold. This will increase the amount of dry gas being mixed with the released sample. Alternatively or additionally, the flow of the second gas may be reduced if the humidity of the dried sample is above the desired threshold. This will also result in an increase in drying by reducing the amount of sample which is released. Alternatively, as mentioned above, the gas flow rates from the sources may be controlled to provide the desired humidity.

The system may further comprise a thermal control module which comprises a sensor to sense the temperature of dried, released sample and a controller to monitor the sensed temperature and adjust the temperature of the mixed dried sample before capture by the recapture device. The temperature may be adjusted to optimise the volume of the mixed dried sample which is captured by the recapture device.

The method and system above transfer the sample (i.e. compounds) which have been collected on the sampler to another device (which may be termed a recapture device) for analysis. The transfer process provides an opportunity to reduce the humidity and/or the temperature of the transferred sample, for example a sample collected using the known devices described in the introduction. The recapture device may comprise one or more sorbent tubes which may have the same sorbent material as the sampler. The mass of the recapture device may be lower (e.g. several times lower) than the mass of the sampler.

Another method of reducing the humidity is to provide a breath sampling device in which humidity can be reduced such as the breath sampling device described above in the first aspect. A sample from such a reduced humidity device may also be subject to further reduction in humidity by using the method and system described above.

### BRIEF DESCRIPTION OF DRAWINGS

For a better understanding of the invention, and to show how embodiments of the same may be carried into effect, reference will now be made, by way of example only, to the accompanying diagrammatic drawings in which:

FIG. 1a is a known breath sampling device in use;

FIG. 1b is an alternative known breath sampling device;

FIG. 1c is a graph of amount of D5 ethanol recovered against relative humidity level for two known adsorbent materials;

FIG. 1d is a flowchart of a process for removing water which is present in the breath sample captured using a device such as that shown in FIG. 1a;

FIG. 2 is a schematic block diagram of the component of a breath sampling device as described herein;

FIG. 3 plots water uptake against relative humidity for two known adsorbent materials;

FIG. 4 plots recovered D5-ethanol amount against water uptake for two types of adsorbent material.

FIG. 5a is a cross-sectional drawing showing the inner components of a breath drying device which can be incorporated in the breath sampling device of FIG. 2;

FIG. 5b is a front view of an inlet face of the drying component of the device shown in FIG. 5a;

FIG. 6a is a cross-sectional drawing showing the inner components of an alternative breath drying device which can be incorporated in the breath sampling device of FIG. 2;

FIG. 6b is a front view of an inlet face of the drying component of the breath drying device shown in FIG. 6a;

FIG. 6c is a cross-sectional drawing showing the inner components of a drying bed of the breath drying device of FIGS. 6a and 6b;

FIG. 6d is a cross-sectional drawing showing the inner components of an alternative breath drying device which can be incorporated in the breath sampling device of FIG. 2.

FIGS. 7a to 7c plot the pressure drop (mbar) by flow rate across the drying bed and across the bypass tubes for three different arrangements of bypass tubes;

FIGS. 8a to 8c plot the estimated drying ratio by flow rate across the drying bed and across the bypass tubes for three different arrangements of bypass tubes;

FIG. 9 is a plot of the experimentally measured pressure drop across the drying bed for a particular arrangement;

FIG. 10 is a flowchart of a method for using the breath sampling device of FIG. 2;

FIG. 11 is a system for drying a breath sample;

FIG. 12 is a flowchart of a method of using the system of FIG. 11.

FIG. 13 is a bar graph of experimentally measured water gain for the sampler and recapture device.

FIG. 14 plots experimentally measured fold-reduction of water from the sampler to a recapture device.

### DETAILED DESCRIPTION

Broadly speaking, we describe a breath sampling device that is configured to collect a breath sample while drying the breath. This can be achieved by drying the breath while collecting the sample. In use, a user exhales into the device (once or several times or even hundreds of times), via a mouthpiece, and the exhaled breath passes through the adsorbent material whereby a large portion of the exhaled breath can be sampled and the desired volatile organic compound(s) within the breath can be adsorbed by the adsorbent material. As an example, the desired volatile organic compound(s) may be D5-ethanol. The breath capture is termed unassisted because the user provides the necessary pressure to push the breath through the device. Therefore, beneficially, the breath sampling device is optimized so that a user can comfortably and easily breathe through the device, and the device can collect as much volatile organic compounds (VOCs) as possible.

As an alternative to or in conjunction with the drying of the device, the breath sample may be dried while transferring it to a plurality of sample tubes (or other recapture devices) after collection. In both cases, the breath sample is dried by mixing the breath with dry gas, e.g. from a flow-controlled gas source. Post collection dehydration is used to improve the collected sample which is typically a relatively large volume of breath. A large volume of breath contains a large amount of water which if not removed, saturates and can even damage an analysis device (e.g. chromatogram column).

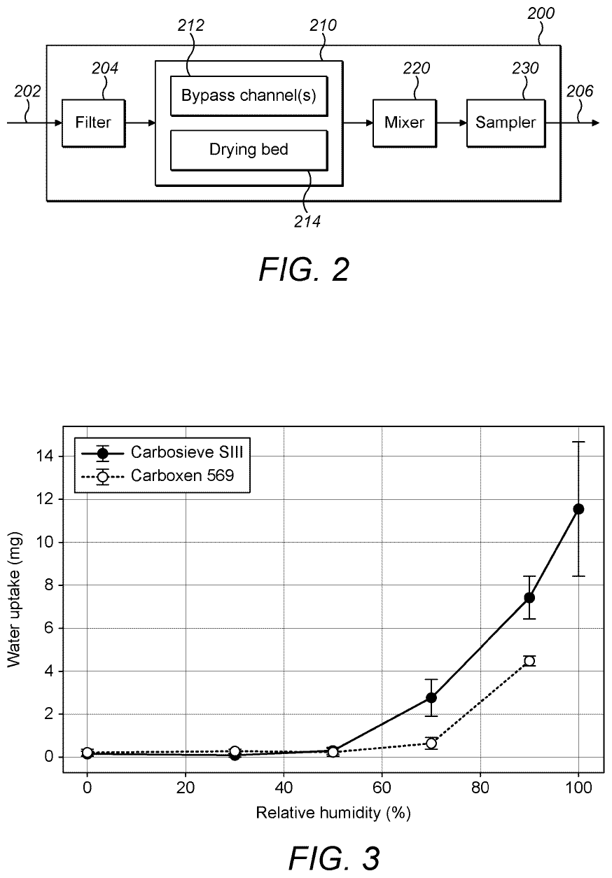

FIG. 2 is a schematic block diagram of the component of a breath sampling device 200 that is configured to collect a breath sample while drying the breath. The breath sampling device 200 comprises an inlet 202 through which exhaled breath passes into the device, an outlet 206 for the exhaled breath and a sampler 230 having a housing in which sorbent material is located. The sampler 230 is connected on a fluid path between the inlet 202 and the outlet 206 so that exhaled breath passes into the device 200, through the sorbent material and out through the outlet 206.

The sorbent material may also be termed an adsorbent material or sorbent, and the terms may be used interchangeably. The sorbent material may be shaped as a disc or any suitable shape and extends across at least a substantial part (at least 50%), up to perhaps the entire, cross-section of the housing. The sorbent material typically samples a substantial part of each exhaled breath. The sorbent material may be made from activated carbon and/or zeolites and/or activated alumina and/or lignite coke and/or bentonite. A suitable material is carbosieve® SIII 60-80 and/or Carboxen® 569. Where appropriate, the sorbent material may be baked, for example in the range of 250 degrees Celsius to 350 degrees Celsius, to condition the sorbent material to remove volatile compounds such as ethanol. The disc (or other shape) of sorbent material is typically removable from the breath sampling device to allow analysis of the volatile organic compounds which are captured in the sorbent material. The parameters of the sorbent material (including diameter and thickness/mass) may be varied to optimise the design of the device, e.g. to ensure that the breath capture is unassisted and breakthrough volume is achieved.

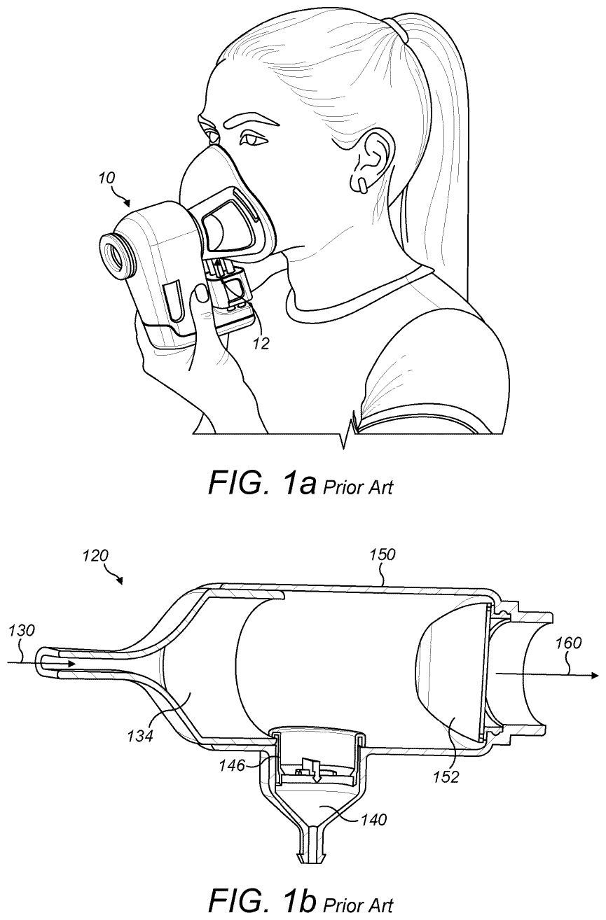

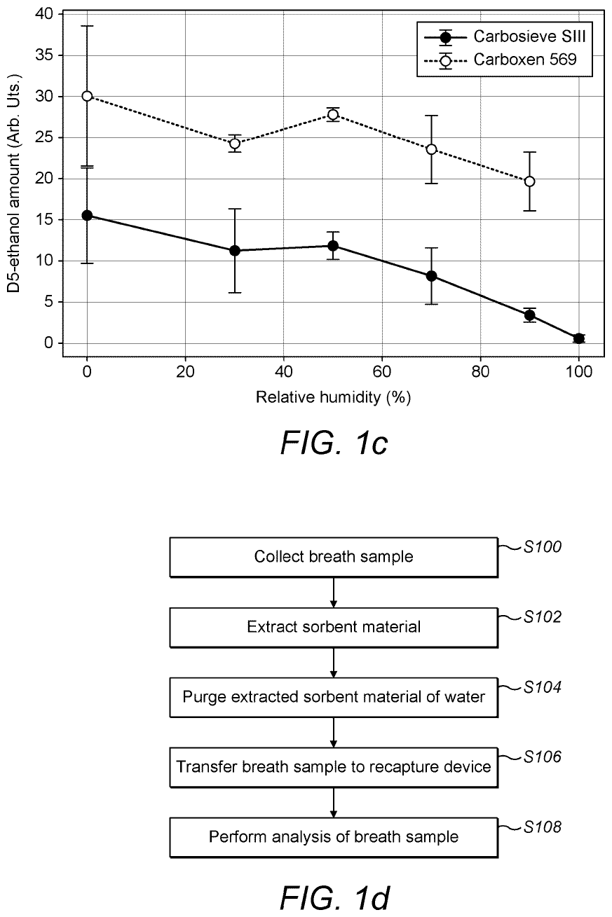

The inlet 202 through which exhaled breath passes into the device and which also acts as an outlet for inhaled breath to a user may be in the form of a mouthpiece. The mouthpiece ensures a non-invasive sampling during normal tidal breathing. In use, the user (who can be a patient), places the mouthpiece in or in the vicinity of their mouth and breathes into the mouthpiece. The user provides the necessary pressure to push the breath through the device 200 thus achieving unassisted breath capture. Alternatively, a mouthpiece that can be placed in/around a human mouth without causing any substantial discomfort to the user may be used (e.g. as shown in FIG. 1a). The mouthpiece may be made of any suitable material, e.g. silicone and/or plastics. The mouthpiece may be detachable so that it can be changed after every use. Thus, beneficially, the same breath sampling device may be used by more than one user/patient.

As shown in this arrangement, an optional breathing filter 204 may be connected after the inlet 202. The function of filter 204 may be to prevent bacteria and viruses entering the device when exhaling from passing through filter 204 to the sorbent material.

The breath sampling device 200 also comprises a drying component 210. The drying component 210 is located between the inlet 202 and the sampler 230, and preferably after the filter 204 when one is used. The drying component 210 is designed to split the fluid path between the inlet and the outlet into a first and a second pathway. In this way, a breath sample from a subject is split into a first portion which passes along the first pathway and is dried in the drying component 210 and a second portion which passes along the second pathway straight through the drying component 210 without being dried. The first and second portions pass through the first and second pathways simultaneously, i.e., at the same time. The first portion may be dried by a drying bed 214 which is housed in the drying component 210. The drying bed 214 may comprise one or more molecular sieve pellets which remove the majority of water from the second portion. The second portion is isolated from the drying bed 214 by passing through one or more bypass channels 212 which do not contact the drying bed 214.

The breath sampling device 200 also comprises a mixer 220 which is located between the drying component 210 and the sampler 230. The mixer 220 recombines the first and second pathways into a single fluid path. Thus, the mixer 220 receives both the first portion after it has been dried and the second portion as it outputs the one or more bypass channels 212. The first portion is preferably dried to a humidity level of between 0 to 10% and the second portion may have a humidity level of between approximately 60 to 80% (i.e. unchanged from the original humidity of the breath sample received through the inlet. The two portions are then mixed together in the mixer 220 to achieve an overall reduced humidity level. The reduced humidity level may, for example, be approximately 40% or any other reduced humidity level at which a target analyte is being adsorbed in sufficient quantities by the sorbent material. The geometry of the drying bed and the bypass channels are such that a set drying ratio with low variation is achieved across a large range of respiratory flows.

FIG. 3 plots water uptake (mg) against relative humidity level (%) at 22° C. at a sample size of 2.1 litres for two types of adsorbent material-carbosieve® SIII 60-80 and/or Carboxen® 569. For both types of material, there is little water uptake provided that the humidity level of the sampled breath is below 40%. Thus, as explained above, the breath sample is split into two portions as described above to achieve this optimum humidity level (or lower). There are various features within the drying component which can be adjusted to optimise the humidity level. These features include the type of molecular sieve (i.e. the material), the mass of the drying bed (i.e. the mass of the molecular sieve), the size of the molecular sieve beads (or other drying material), the pore size of the molecular sieve beads and/or other aspects of the geometry of the molecular sieve.

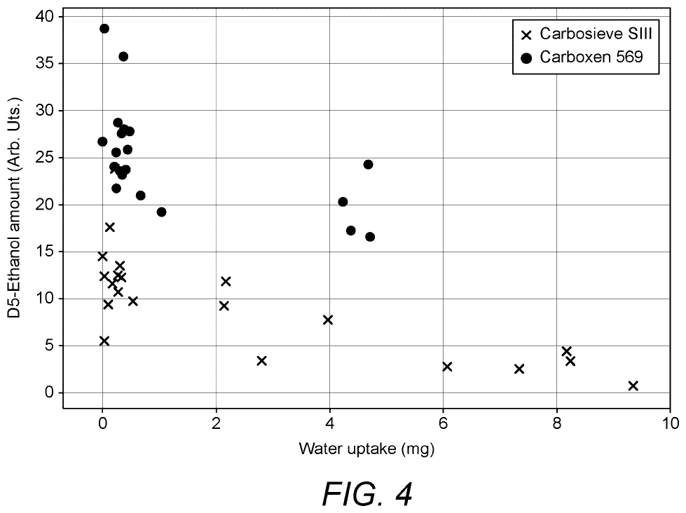

FIG. 4 plots recovered D5-ethanol amount (arbitrary units) against water uptake (mg) for two types of adsorbent material-carbosieve® SIII and/or Carboxen® 569. 100 mg of each adsorbent material was exposed to a sample volume of 2.1 litres delivered at a rate of 200 ml/min through a standard quarter-inch sorbent tube. It can be seen that Carboxen® 569 appears to adsorb higher amounts of D5-ethanol with lower levels of water uptake, thus making it a more suitable adsorbent material for the use cases described in the present application.

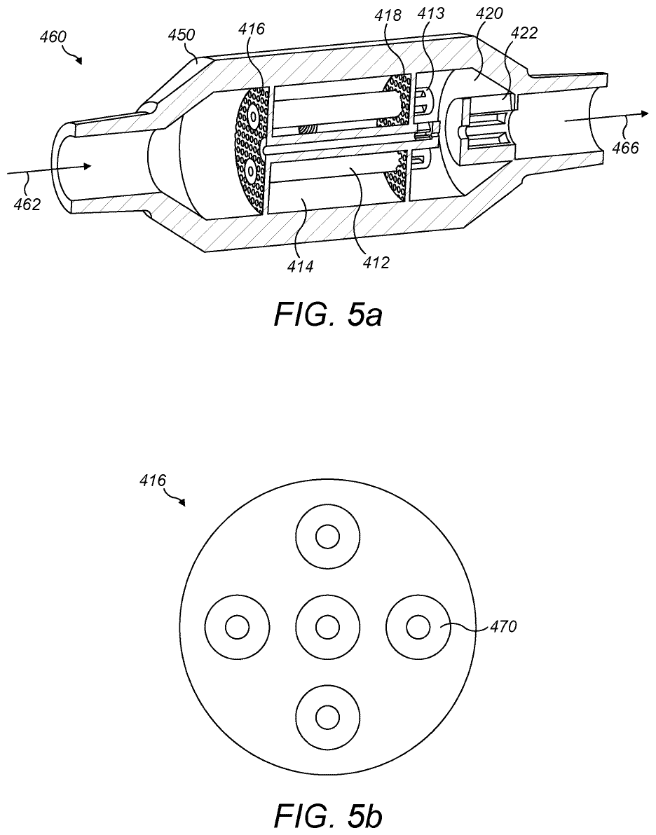

FIG. 5a is a schematic diagram of one example of a component which can be used in the breath sampling device of FIG. 2. The component in FIG. 5a incorporates both the drying component and the mixer shown in FIG. 2 and thus may be termed a humidity optimiser 460. It will be appreciated that the drying component and the mixer could be designed as separate sub-components or they could be integrated as shown in FIG. 5a.

The humidity optimiser 460 comprises a housing 450 have an inlet 462 and an outlet 466. The housing 450 may be made of plastic or any suitable material. The housing 450 is generally in the form of a cylindrical hollow tube which tapers at the inlet and the outlet. The inlet 462 is connected to the outlet of the filter (when one is used) and the outlet 466 is connected to the sampler. A sampled breath thus flows through the inlet 462 into the drying component, into the mixer 420 and out of the outlet 466 to the sampler. The drying component is in the form of a molecular sieve which comprises a pair of inlet and outlet faces 416, 418 (which may also be termed plates). Each inlet and outlet face 416, 418 is generally disc-shaped and extends across the cross-section of the housing 450. In this arrangement, the direction of flow of an exhaled breath through the housing 450 is approximately perpendicular to the plane of each face. Drying particles (not shown) are housed in the drying bed 414 between the plates 416, 418 to dry the first portion of air.

The inlet and outlet plates 416, 418 comprise a mesh having a plurality of pores and the mesh surrounds a plurality of holes (may also be termed apertures). Each hole on the inlet face of the drying component is connected to a corresponding hole on the outlet face by a bypass tube 412 which extends along the housing through the drying component. The first portion of the breath sample flows through the mesh into the drying component and is dried by the drying material in the drying bed 414. At the same time, the second portion of the breath sample flows through the holes and through the bypass channels, thus bypassing the drying material and is not changed.

As shown more clearly in FIG. 5b, in this example, there are five holes 470 in the inlet plate 416 but it will be appreciated that the number is adjustable to optimise the design as described below. For example, there may be a single hole. The diameter of each hole may be small compared to the diameter of the face, for example less than 10% of the diameter, and the overall cross-sectional area of the holes may be approximately 30% of the cross-sectional area of the face. The holes may have the same or different diameters. For example, a central aperture may have a larger diameter than apertures positioned towards the edge of the face. Each hole 470 may be tapered with a larger diameter on the external side and a smaller diameter on the internal (i.e. drying bed) side. Merely as an example, the external side diameter may be 2.5 mm and the internal side diameter may be 2.05 mm so that there is a tapering of approximately 20%. In this arrangement, each hole is surrounded by a mesh. There may be a central hole and the other holes may be equally spaced around the central hole. The holes are spaced apart to allow a sufficient volume of air to pass through the mesh between the holes into the drying beds. As an example, the spacing between each hole may be at least 4 mm for a sieve diameter of 30 mm.

Returning to FIG. 5a, the drying particles may be any suitable particle and may have diameters of between 1 mm to 5 mm with an average diameter of 3 mm. The length of each bypass tube is the same as the length of the molecular sieve and may be approximately the same or slightly longer than the diameter of each plate. For example, the length may be 40 mm.

The mixer 420 comprises a chamber positioned between the drying component and the outlet 466. The first and second portions of the breath sample initially mix within the chamber. By using a plurality of bypass tubes, mixing of the first and second portions of the breath sample within the chamber may be encouraged. The plurality of bypass tubes are evenly spaced across the cross section of the plate which may also facilitate mixing of the first and second portions. In this arrangement, the bypass tubes extend beyond the outer plate 418 and comprise apertures in the protruding portion to form a dispersive element 413 which may further aid with mixing. The mixing of the first and second portions of the breath sample that takes place in the chamber may be considered a pre-mixing stage. Additionally or alternatively, further mixing is encouraged by using a mixing component 422 which comprises a channel which has a smaller diameter than the chamber. The breath sample which is partially mixed in the chamber is then combined more thoroughly in the mixing component 422. The mixing component 422 used may be a standard mixing component, such as a static mixer.

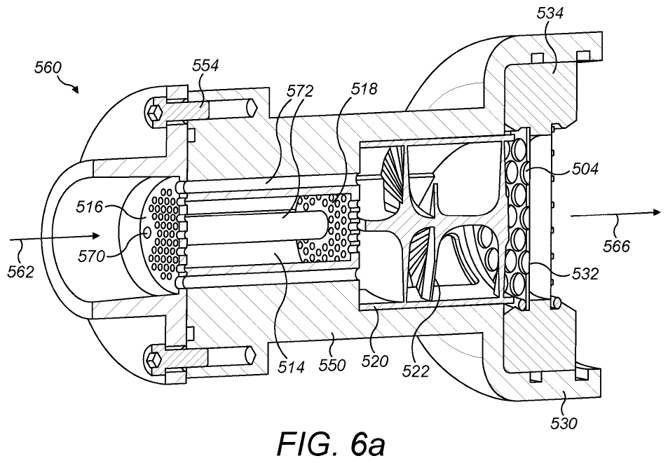

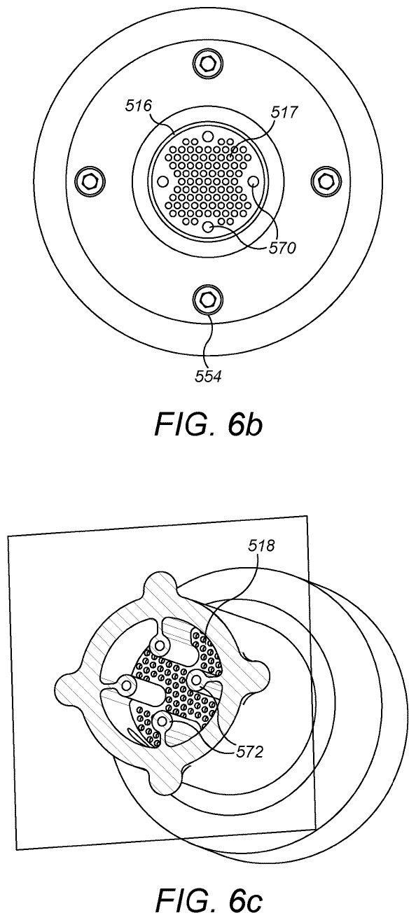

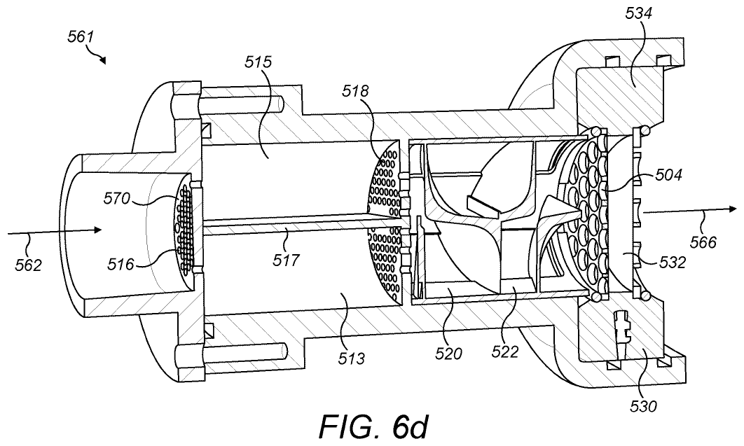

FIGS. 6a and 6b show an alternative breath sampling device 560 comprising a housing 550 having an inlet 562 and an outlet 566. As in the previous embodiment, the housing 550 is generally in the form of a cylindrical hollow tube but in this example, the housing 550 houses both the humidity optimiser and the sampler 530. The sampler 530 is housed in a portion of the housing with a larger diameter than the rest of the housing. The sampler 530 comprises a filter 504 in the form of a mesh over a collection disc 532 which is held in a support 534. In this example, the housing is not a single integral housing and the sampler 530 is in a second portion of the housing which is attached to the main portion of the housing using fixings 554.

As in the previous embodiment, within the housing there is a drying component and a mixer 520. A sampled breath flows through the inlet 562 into the drying component, into a mixer 520 and out of the outlet 566 to the sampler. The inlet is designed to be compatible with standard mouthpieces, for example by using an inlet having a diameter which conforms to the relevant standards, e.g. a 15 mm or 22 mm diameter inlet. The drying component is in the form of a molecular sieve which comprises a pair of inlet and outlet faces 516, 518 (which may also be termed plates or retainers). Drying particles are housed between the plates 516, 518 to dry the second portion of air in a drying bed 514. Merely as an example, the cavity in which the drying particles are housed may be 30 mm in diameter and 38 mm in length. Each inlet and outlet face 516, 518 is generally disc-shaped and extends across the cross-section of the housing 550. In this arrangement, each plate is in the form of a continuous mesh having a plurality of apertures 570

As shown more clearly in FIG. 6b, in this arrangement, a plurality of holes 570 are arranged around the mesh 517 towards the outer edges of the inlet face 516. The arrangement is the same on the outlet face 518. As shown in FIG. 6c, bypass tubes 572 connect the holes on the inlet and outlet faces 516, 518. It will be appreciated that other arrangements of mesh and holes may be used. For example, the whole surface of each face may be in the form of a continuous mesh or only parts of the face may comprise a mesh. Holes may be arranged within or around the mesh. Bypass tubes may align with holes or with the mesh. Each bypass tube is made from a material (e.g. plastic) and is relatively thin but strong enough to provide structural stability. For example, each bypass tube 572 may have an outer diameter of 2.5 mm and an inner diameter of 2 mm (i.e. the tube wall has a thickness of 5mm). In this arrangement there are four bypass tubes which are equally spaced towards the edge of the drying bed (i.e. towards the edge of the inlet and outlet faces).

Returning to FIG. 6a, in this arrangement, the mixer 520 comprises a mixing component 522 in the form of a helical static mixer. In this example, the mixing component 522 comprises a series of baffles. There are two streams of breath which are input into the static mixer and which are blended as the breath sample moves through the mixing component 522.

FIG. 6d shows an alternative breath sampling device 561 which has many features in common with the arrangement of FIG. 6a and thus the same reference numbers have been used for the same features. As in the previous arrangement, the breath sampling device 561 comprises a housing 550 having an inlet 562 and an outlet 566 and the housing 550 houses both the humidity optimiser, the sampler 530 and the mixer 520. As in the previous arrangement, the drying component is in the form of a molecular sieve which comprises a pair of inlet and outlet faces 516, 518 (which may also be termed plates or retainers) and in this arrangement are also in the form of plates having a plurality of apertures 570. However, in this arrangement, a divider 517 is connected between the inlet and outlet faces 516, 518 to separate the drying component into two chambers 513 and 515 which are separated from one other by the divider 517. The first chamber 513 comprises drying material to form the drying pathway and acts as the molecular sieve. The second chamber 515 does not contain drying material and thus provides the second pathway and may be considered a bypass channel.

The first and second chambers may be identical in size and may thus also referred be to as halves. The identical size of chambers to achieve a constant split-ratio across all flow rates of 50:50. It will be appreciated that the relative size of each chamber may be adjusted to achieve a desired split-ratio across all flow rates. Each of the first and second chambers is filled with materials which are mechanically identical (bead size and geometry), but chemically different. The first chamber 513 is the drying cavity and comprises material which removes water from the airstream. The second chamber 515 comprises chemically inert material which does not alter the airstream. As described above, the drying material may comprise a bed of drying particles in the form of beads of a molecular sieve. The material which is chemically inert may be glass beads. The glass beads may be chemically inert and have a similar size and geometry as the drying particles. In this way, the first portion of air which passes through the first chamber is dried and the second portion of air which passes through the second chamber is not dried, but the two portions of air would have the same pressure flow characteristics across all flow-rates and hence a constant split ratio. Glass beads may also be included with the drying particles in the drying bed.

As explained above, it is important that the ratio of pressure drop across the drying bed and pressure drop across the bypass channel(s) is relatively constant, so that the same ratio of air is maintained through the drying component to the mixer.

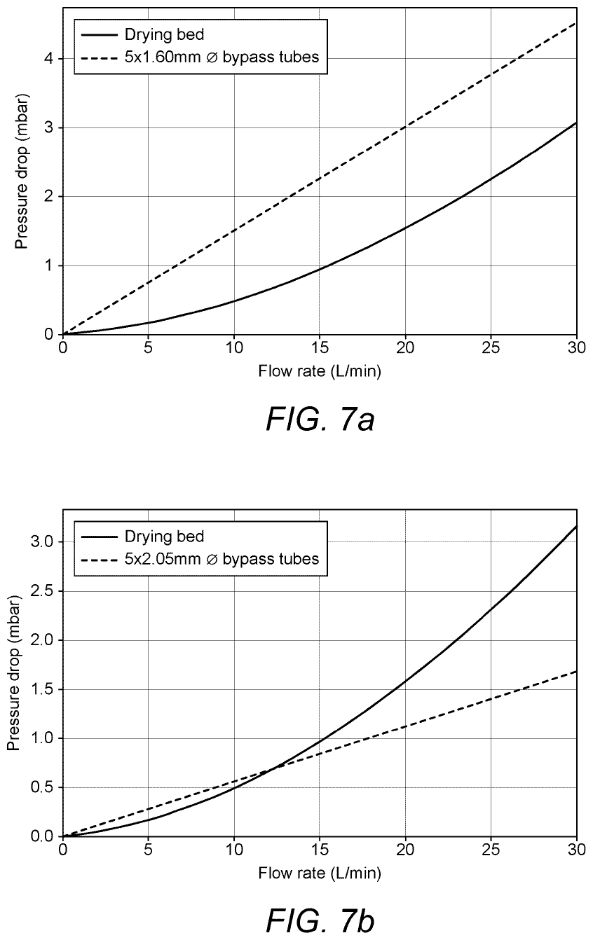

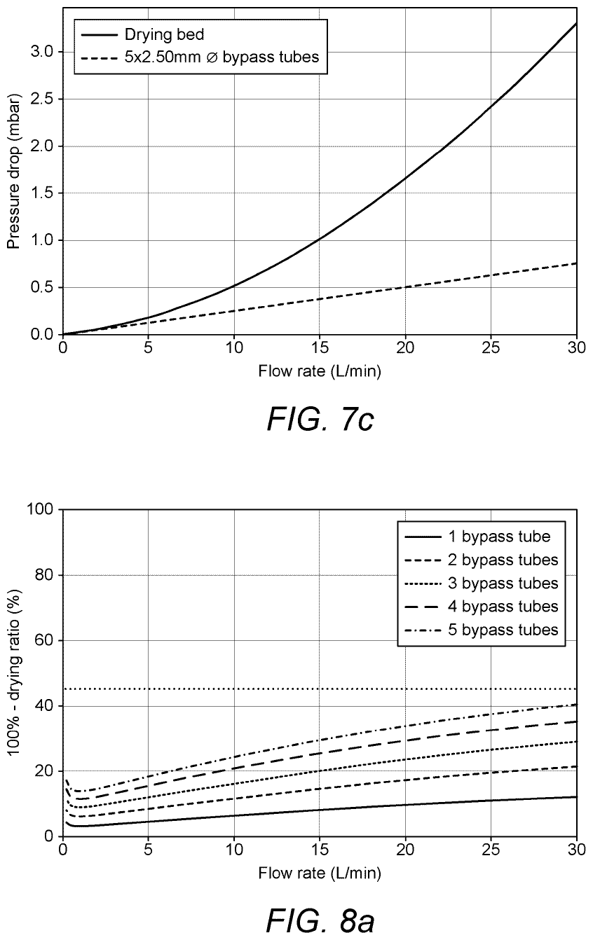

FIGS. 7a to 7c each plot the pressure drop (mbar) across the drying bed and across the bypass tubes as a function of flow rate to show the variation in pressure drop caused by changing the diameter of the bypass tubes. In each example, the length is maintained at 40 mm, the nature of the drying bed is unchanged and there are five bypass tubes.

FIG. 7a compares the simulated pressure drop across 5 tubes of 1.6 mm diameter with the pressure drop across the drying bed. FIG. 7b compares the simulated pressure drop across 5 tubes of 2.05 mm diameter with the pressure drop across the drying bed. FIG. 7c compares the simulated pressure drop across 5 tubes of 2.5 mm diameter with the pressure drop across the drying bed. In FIGS. 7a to 7c the drying bed is filled with uniform beads of size 2 mm, which act as a molecular sieve. The plots shown in FIG. 7b are the most consistent which suggests that, of the diameters which are plotted, the optimum diameter is 2.05 mm when using 5 holes and bypass channels of length 40 mm. It will be appreciated that similar plots can be prepared for different lengths of channels and different numbers of holes to optimise other features. Similarly, the drying bed can be optimised in a similar way.

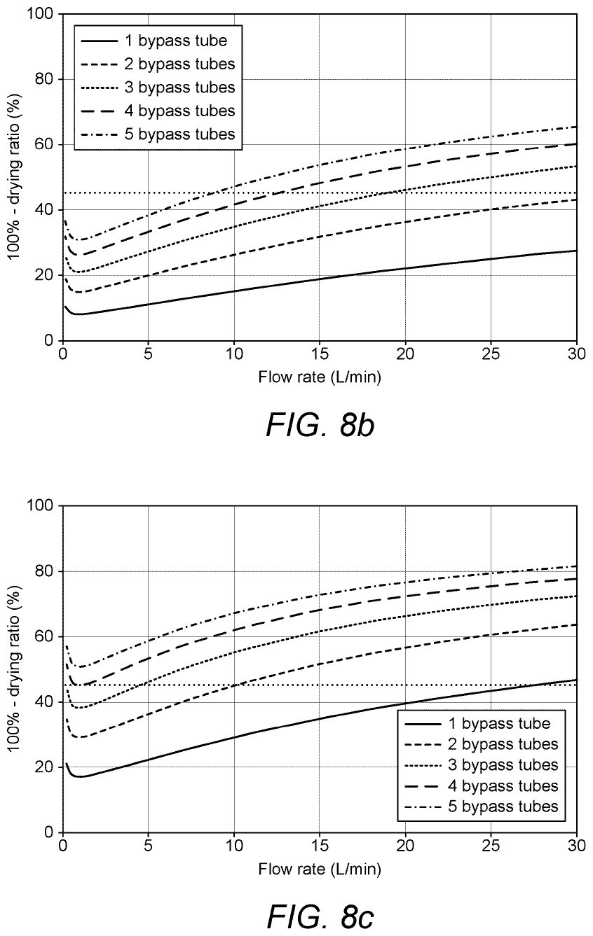

As explained above, a target humidity level for the mixed output at which a target analyte is being adsorbed in sufficient quantities by the sorbent material, such as, for example, 40% humidity for the mixed output, is desired. The features which can be adjusted to optimise the humidity level, include the type of molecular sieve (i.e. the material), the mass of the drying bed (i.e. the mass of the molecular sieve), the size of the molecular sieve beads, the pore size of the molecular sieve beads and/or other aspects of the geometry of the molecular sieve. Humidity levels may also be affected by the diameter, length and number of the bypass channels. Merely as an example, FIG. 8a to 8c plot the simulated variation in output humidity, i.e. 100% of the initial humidity minus the drying ratio, as a function of flow rate for different numbers of tubes. In each Figure, there may be between 1 to 5 of the tubes which are open. In each example, the length is maintained at 40 mm and the nature of the drying bed is unchanged.

FIG. 8a compares the simulated output humidity when using between 1 and 5 tubes of 1.6 mm diameter. FIG. 8b compares the simulated output humidity when using between 1 and 5 tubes of 2.05 mm diameter. FIG. 8c compares the simulated output humidity when using between 1 and 5 tubes of 2.5 mm diameter. The plots shown in FIG. 8b are the closest to a possible target of 40% which suggests an optimum diameter of 2.05 mm when using 5 holes and bypass channels of length 40 mm for reaching a 40% target humidity. It will be appreciated that similar plots can be prepared for different lengths of channels and different numbers of holes to optimise other features. Similarly, the drying bed can be optimised in a similar way. Additionally, the optimum diameter will depend on the bead size of the molecular sieve used. In FIGS. 8a to 8c the beads were uniform with a diameter of 2 mm. It will be appreciated that these plots will therefore change if the properties of the molecular sieve that is being used change.

FIGS. 7a to 8c are simulations which can be used to identify optimal parameters of the breath sampling device to achieve the desired flow rate and humidity. Once the parameters are selected, a breath sample device may be tested to see whether the simulations are accurate.

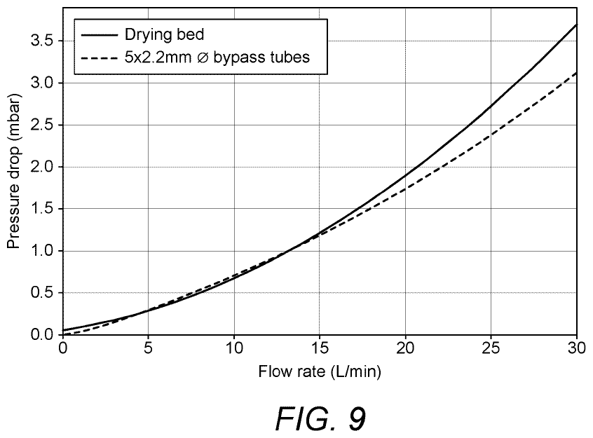

FIG. 9 plots the experimentally measured pressure drop with the pressure drop across the drying bed for a breath sampling device of the design described above. The drying component comprises five bypass tubes each having a 2.2 mm internal diameter and a length of 38 mm. The bypass tubes are located inside a cylindrical drying bed region with a diameter of 30 mm and a length of 38 mm. The experimentally obtained static-pressure curves of FIG. 9 show a more favourable ratio across a larger range of flows than the simulated static-pressure curves previously described. In addition to experimentally measuring the pressure drop, the drying performance at a flowrate of 5 l/min was measured. The drying performance was 50% which is in agreement with the static-pressure data shown in FIG. 9.

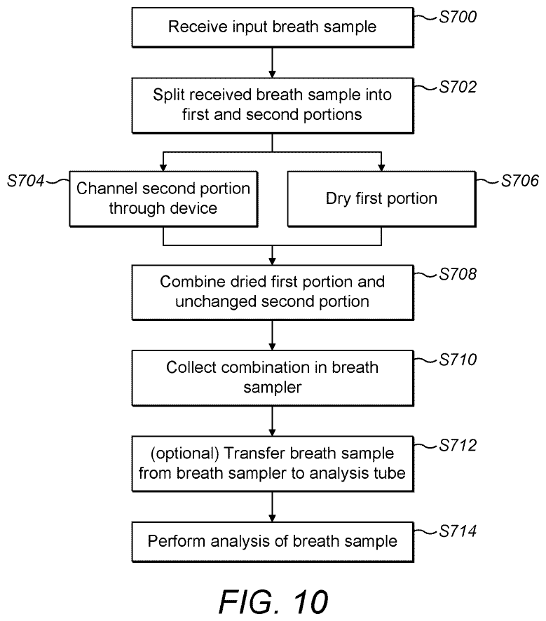

FIG. 10 is a flowchart showing how a breath sample which is collected by a breath sampling device such as FIG. 2 may be processed. In a first step S700, an input breath sample is received. The breath sample is split into first and second portions at step S702 and the second portion is channeled through the device at step S704 without any alteration, in particular to the humidity level. Simultaneously, the first portion is dried to remove at least some of the water content at step S706. Steps S702 to S706 may be achieved for example using the drying component with a drying bed and bypass channels as described in FIG. 5a. The first and second portions are then recombined so that the overall humidity of the breath sample is reduced but the proportion of the first and second portion is unchanged at step S708. The breath sample may now have an optimised level of humidity for collection in the breath sampler at step S710.

The collected breath sample is then removed from the breath sampling device for analysis, e.g. by removing the disc of adsorbent material and sending this to a separate laboratory. The breath sample which has been captured on the disc may be transferred to another device for ease of analysis. For example as shown at step S712, the breath sample may optionally be transferred to a sorbent tube such as those shown in FIG. 1a. At step S714, the breath sample is then analysed to determine the quantity of the target volatile organic compound (may also be termed a volatile reporter) therein. This analysis may be done in any suitable apparatus, e.g. a mass spectrometer or other analyser.

It will be appreciated that some of the VOC may be lost during any or all of the following steps: when collecting the sample at step S710, when sending the breath sample (on its sampler) to a laboratory, when transferring at step S712 the sample from the sampler to a standard sorbent tube (or the like) and when analysing at step S714. However, there is no step of removing the water itself from the disc and thus there should be a reduction in the loss of the VOC when compared to other processes which use this step. The largest loss of VOC is typically when purging water from the disc which contains the breath sample.

The target compound (which may also be termed a volatile reporter) may be a compound containing a hydroxyl group. The volatile reporter may be selected from methanol, ethanol, propanol, isopropyl alcohol, isobutyl alcohol, butyl alcohol, 2-methyl-3-buten-2-ol, 1-penten-3-ol, isoamyl alcohol, amyl alcohol. The volatile reporter may be labelled as discussed above for example D3-methanol, D5-ethanol, D7-propanol, D7-isopropyl alcohol. The volatile reporter is produced when a volatile functional group is cleaved from a glycoside via cleavage of the glycosidic bond. For example the volatile functional group may be selected from methyl, ethyl, propyl, isopropyl, butyl, methyl-D3, ethyl-D5, propyl-D7. More specifically, when the volatile functional group ethyl-D5 is cleaved from the glycoside it produces D5-ethanol.

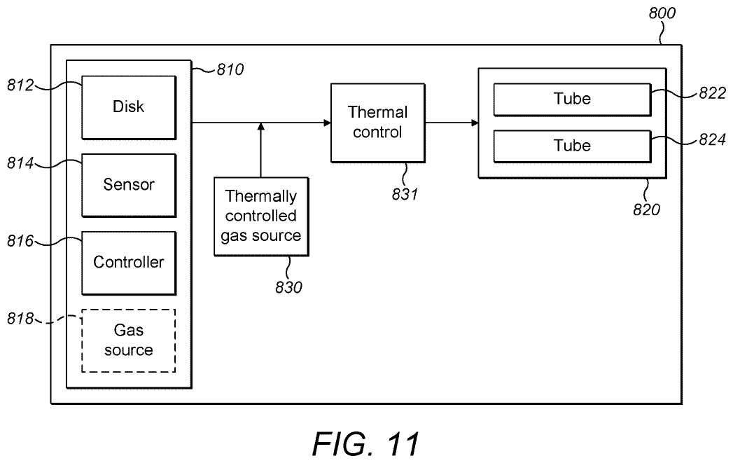

It will be appreciated that the drying components of the breath sampling devices described above may not bring the humidity below the desired humidity threshold and in such cases, there is a need to reduce the humidity by a further small amount (e.g. 5-10%). FIG. 11 is a system 800 which can be used together with the breath sampling device of FIG. 2 to achieve an optimum humidity level. It will also be appreciated that the system 800 of FIG. 11 may be used with a standard breath sampling device such as the one shown in FIG. 1a.

The system 800 comprises a chamber 810 into which a disk 812 of adsorbent material is placed after being removed from the breath sampling device. The chamber 810 may be nitrogen chamber, i.e. filled with nitrogen so that there is no humidity present in the chamber. The disk may be heated in the chamber 810 so that the disk releases any volatile organic compounds and water which have been captured in the disk whilst it was in the breath sampling device. The chamber 810 may be fed with dry nitrogen so that there is a flow through the disk to encourage the gases to be released. The released gases exit the chamber 810 as indicated by the arrow.

The system 800 also comprises a thermally and flow-rate controlled gas source 830 which generates a flow of dry gas which is to be mixed with the released gases before the mixture is captured by a recapture device 820. The gas source 830 may generate a flow of dry nitrogen at a desired flow rate. After the gas source is mixed, it may be thermally controlled again 831. By thermally controlling the mixing gas, the formation of aerosol droplets that may be long-lived after reheating the gas may be prevented. The thermal control may comprise at least one sensor sensing the temperature and a mechanism for controlling the temperature of the mixed gas in relation to the sensed temperature. Any suitable sensor and thermal control may be used.

The recapture device 820 may comprise a plurality of sorbent tubes 822, 824. By tube, it is meant any holder with sorbent material. The sorbent tubes may be carbosieve® SIII 60-80 tubes, carboxen® 569 tubes or any similar, suitable sorbent tube or holder. By using multiple recapture devices, multiple samples may be captured, either for back-up purposes or to repeat analysis.

The system 800 also comprises an optional second flow-rate controlled gas source 818 which generates a flow of a second gas through the sorbent material while the sorbent material is being heated. The second gas source 818 is shown the chamber 810 but the gas source may be located outside the chamber 810. When the first and second gases are the same, e.g. dry nitrogen, a single source may be used to generate both flows. It will be appreciated that increasing the flow rate of the second gas through the disk will speed up the transfer process but the humidity must be kept below a target threshold.

The target threshold may be, for example, approximately 40% or any other reduced humidity level at which a target analyte is being adsorbed in sufficient quantities by the sorbent material. Alternatively, when used with the breath sampling devices described above, the reduction in humidity level may only be a small reduction, perhaps 5-10%. The humidity of the recaptured breath sample is dependent on the humidity of the gases released from the disc and the ratio of the flow rate from the disk to the gases generated by the gas source. Humidity could be controlled using an active control (i.e. closed loop) with a humidity sensor 814 and one or more flow controllers 816. For ease of the flow controller 816 and sensor 814 are shown within the chamber 810 but it will be appreciated that additional controllers and sensors can be used throughout the system, e.g. to monitor the flow rate at the gas source.

Increasing the flow rate means that the breakthrough volume for the recapture device will be reached more quickly. The breakthrough volume may be defined as the retention volume of a specific compound or compounds by the sorbent material. In other words the retention volume may be the volume of a specific compound or compounds which is actually sampled by the sorbent material as sample is recaptured. The breakthrough volume is also increased by a larger volume recapture device and decreasing the temperature of the mixed gases. Thus, the breakthrough volume can be increased by swapping the sorbent tubes every 15 minutes (or other fixed time) so that a larger number of tubes can be used. Additionally or alternatively, several sorbent tubes can be used in series such that the overall capacity of sorbent material is sufficient to adsorb the whole of the collected breath sample without swapping the sorbent tubes, or such that the sorbent tubes need to be swapped less frequently.

Merely as an example, the disk may contain a mixture of water and air which weighs approximately 196 mg and there may be 5 nanograms of the target VOC (D5 ethanol). The heating process releases gases having approximately 83 mg of water per litre at 50 degrees Celsius when a flow rate of 50 ml/min is maintained over the disk. The gas source generates a flow of dry nitrogen (0% humidity) at room temperature (say 22 degrees Celsius), with a flow rate of 480 mL/min. The mixture of released gases and generated gases thus has a temperature which is approximately at room temperature and contains 5.8 mg of water/L (i.e. 30% humidity). The humidity of the mixed gases is thus below the target threshold of 40%. The tubes can be swapped so that a total of 10 tubes are used. In this way, the total recovery of the target VOC (D5 ethanol) was over 70%.

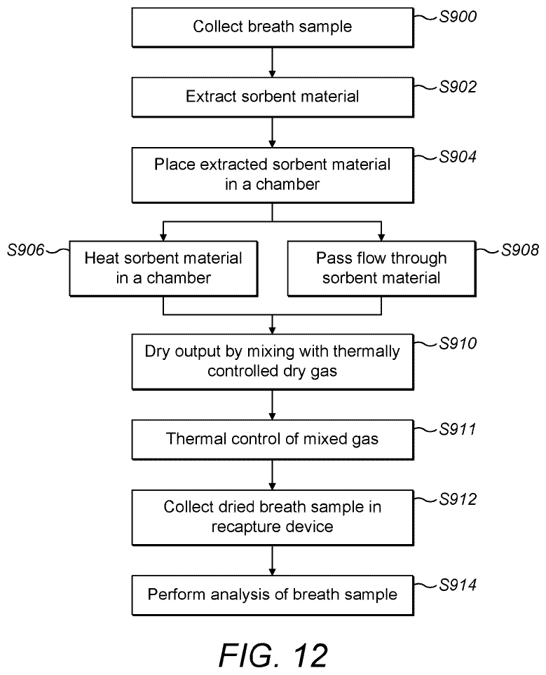

FIG. 12 is a flowchart of a method of using the system of FIG. 11. In a first step S900, a breath sample is collected, for example using the device of FIG. 2 but more normally by using a breath sampling device without a drying component. At step S902, the sorbent material (also termed sampler) is extracted. The sorbent material may be in the form of a disc or any other shaped component or may be included in a disc or any other shaped holder. The extracted sorbent material with or without its holder is then placed in a chamber of the system at step S904. The system may for example be in a laboratory.

At step S906, the sorbent material is heated to release any gases captured on the material. Thus, the sorbent material may be heated in a range which covers the desorption temperature of the gases captured on the material, e.g. between 180° C. to 250° C. for ethanol and more generally between ambient temperature and the maximum temperature of the sorbent material, e.g. 350° C. At step S908, a flow of dry gas (e.g. dry nitrogen) may optionally be passed through the sorbent material to encourage the flow of the released gases. The released gases are output from the chamber and mixed with a dry gas to reduce the humidity at step S910. At step S911 the temperature of the mixture of the released gases and the dry gas is thermally controlled. Although steps S906, S908, S910 and S911 are shown as separate steps they may in effect be done simultaneously so that at step S912 a dried breath sample is captured in the recapture device (which as described above may comprise one or multiple devices). In other words, the breath sample is transferred from the sorbent material (e.g. disk) to the recapture device (e.g. sorbent tube) whilst the breath sample is simultaneously heated and diluted with dry gas.

The recapture device may comprise at least one a sorbent tube which has a temperature, humidity and gas volume dependent capture efficiency for the VOCs of interest, for example D5-ethanol. By simultaneously heating the released gas and mixing it with dry gas, the humidity of the sample which reaches the recapture device can be optimised. Similarly, by thermally controlling the temperature of the mixture, the temperature of the sample may be optimised. As explained above, the volume may be controlled by controlling the flow rate(s) of one or all of the gases within the system and using time to calculate the volume. Suitably, the highest capture efficiency for the VOC of interest is achieved. The recapture device may be then analysed using any suitable device at step S914.

It will be appreciated that some of the VOC may be lost during any or all of the following steps: when collecting the sample at step S902, when sending the breath sample (on its sampler) to a laboratory, when transferring the sample from the sampler to a standard sorbent tube (combined steps S906 to S912) and when analysing at step S914. Although there is drying of the breath sample, there is no separate purging step in which water is removed from the sorbent material prior to transfer and thus there should be a reduction in the loss of the VOC when compared to other processes which use this step. The largest loss of VOC is typically when purging water from the sorbent material which contains the breath sample.

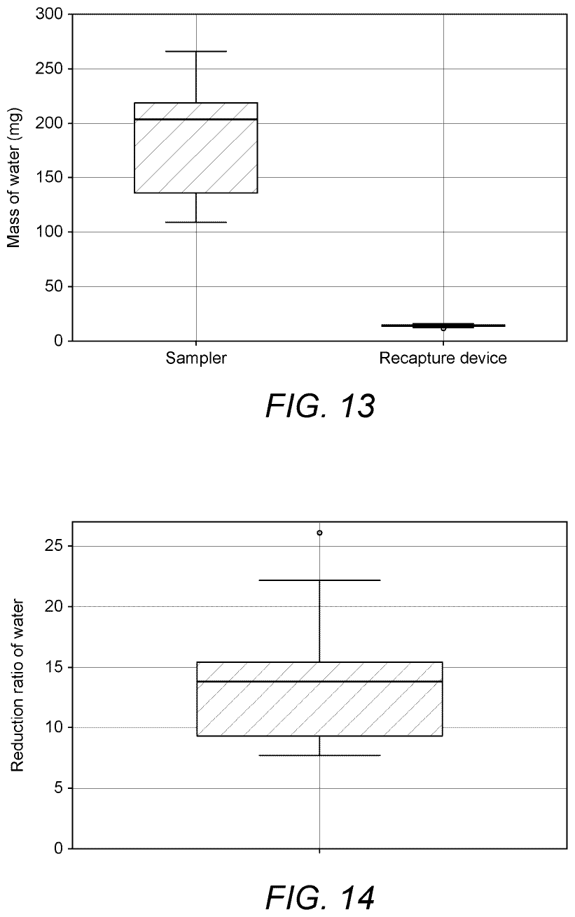

FIG. 13 plots experimentally measured water gain for the sampler and recapture device (a pair of sorbent tubes) for 9 patient breath samples. The average sample volume was 41.6 L and the average water uptake by the sampler was 202 mg. After transferring the breath sample to the recapture device (in this case a pair of sorbent tubes, with the same sorbent material as the sampler, but four times less mass) only 14.5 mg of the water remains. This demonstrates that post collection dehydration results in drier samples. FIG. 13 also demonstrates that the mass of water collected from the recapture device is more consistent, as illustrated by the narrow error bars for the data collected by the recapture device.

FIG. 14 plots the experimentally measured fold-reduction of water from the sampler to recapture device on a per-sample basis. As shown, the amount of water transferred to the recapture device from the sampler is reduced by an average of 14 fold. In both FIGS. 13 and 14, the coefficient of variance in the samples is 30% and in the recapture device (two tubes), the coefficient of variance is 8%.

In addition to the data in FIGS. 13 and 14, further laboratory testing has been carried out to measure the recovery accurately across the ranges of flow rates and volumes for the VOC of interest, e.g. D5-ethanol. In the testing, D5-ethanol has been sampled in 100% humid air across a range of flow rates (6 L/min to 9.7 L/min), with D5-ethanol amounts (300 pg-500 pg), and sample volumes (30 L-60 L). The transfer process from sampler to recapture device has an efficiency of 60.0% with a coefficient of variance of 18.9%. Under favourable fixed sampling conditions, for example 300 pg of D5-ethanol in 100% humid air being sampled at 9.7 L/min for a total of 30 L, the transfer efficiency can be 72.1% with a coefficient of variance of 12.7%. It is noted that the sampling device of FIGS. 2 to 6d also provides similar operating results.

The contents of all such papers and documents referenced in this specification are incorporated herein by reference.

Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others. All of the steps of any method or process so disclosed, may be combined in any combination, except combinations where at least some of such features and/or steps are mutually exclusive.

1. A breath sampling device comprising:
an inlet through which a breath sample is received;
an outlet through which the breath sample exits the breath sampling device;
a fluid path between the inlet and outlet;
a drying component which is positioned in the fluid path and which is configured to split the fluid path into first and second pathways, whereby, in use, a first portion of the breath sample passes through the first pathway and a second portion of the breath sample passes through the second pathway simultaneously;
wherein the first pathway comprises drying material to dry the first portion of the breath sample; and
wherein the second pathway is separated from the first pathway and the drying material so that the second portion of the breath sample passes through the second pathway without any drying;
a mixer which is positioned in the fluid path downstream from the drying component and which is configured to recombine the first and second pathways into one fluid path, whereby the dried first portion of the breath sample and the second portion of the breath sample are mixed in the mixer to produce an output breath sample with lower humidity than the breath sample received in the inlet; and
a sampler which is positioned in the fluid path downstream from the mixer and which is configured to collect a target compound from the output breath sample from the mixer.

2. The breath sampling device as claimed in claim 1, wherein the first and second pathways are configured so that a ratio of a pressure drop across the second pathway and a pressure drop across the first pathway is constant across a range of flowrates, whereby the same amount of breath sample is maintained through the first and second pathways across the range of flowrates.

3. The breath sampling device as claimed in claim 1 or claim 2, wherein the drying component comprises an inlet face, an outlet face and a drying bed of the drying material held between the inlet face and the outlet face, wherein at least a portion of each of the inlet and outlet faces comprises a mesh.

4. The breath sampling device as claimed in claim 3, wherein the drying material comprises multiple sieve pellets.

5. The breath sampling device as claimed in claim 4, when dependent on claim 2, wherein when configuring the first pathway, the parameters which are configured comprise at least one of a mass of the drying material, a size of each sieve pellet, pore size of the sieve and other aspects of the geometry of the sieve.

6. The breath sampling device as claimed in any of claims 3 to 5, wherein the drying component comprises at least one channel which connects the inlet face to the outlet face and which is separated from the drying bed.

7. The breath sampling device as claimed in claim 6, wherein the inlet and the outlet face comprise an aperture which aligns with the at least one channel.

8. The breath sampling device as claimed in claim 7, wherein each aperture is larger on an upstream side of the inlet face than the downstream side of the inlet face and each aperture is smaller on an upstream side of the outlet face than the downstream side of the outlet face.

9. The breath sampling device as claimed in any one of claims 6 to 8, wherein there are a plurality of channels.

10. The breath sampling device as claimed in claim 9, comprising a central channel which is covered by the mesh on each of the inlet and outlet faces.

11. The breath sampling device as claimed in any one of claims 6 to 10, when dependent on claim 2, wherein when configuring the second pathway, the parameters which are configured comprise at least one of a length of each channel, a cross-sectional area of each of channel and the number of channels.

12. The breath sampling device as claimed in any one of the preceding claims, wherein the drying component comprises a first chamber and a second chamber which are separated from one another and the first chamber comprises drying material to form the first pathway and the second chamber forms the second pathway.

13. The breath sampling device as claimed in any preceding claim, wherein the mixer comprises a static mixer.

14. A method for preparing a collected sample from a breath sampling device according to any one of the preceding claims for analysis, the method comprising:
extracting sorbent material containing the collected sample from the breath sampling device;
placing the sorbent material in a chamber;
simultaneously releasing and drying the collected sample from the sorbent material by:
heating the sorbent material in the chamber to release the collected sample; and
mixing the released collected sample with a dry gas to dry the released collected sample; and
capturing the dried released collected sample in a recapture device which is suitable for analysis.

15. The method as claimed in claim 14, further comprising flowing a second gas through the sorbent material while the sorbent material is being heated.

16. The method as claimed in claim 15, further comprising controlling the temperature of the dried released sample to optimise capture in the recapture device.

17. A system for preparing a collected sample from a breath sampling device according to any one of the preceding claims for analysis, the system comprising:
a chamber which is configured to receive sorbent material containing the collected sample from the breath sampling device,
a heat source for heating sorbent material received in the chamber to release the collected sample from the sorbent material;
a first gas source for providing a flow of dry gas which is mixed with the released sample as the sample is released, whereby the sample is simultaneously dried and released from the sorbent material; and
a recapture device which is configured to capture, for analysis, the sample which has been simultaneously dried and released.

18. The system as claimed in claim 17, wherein the chamber is filled with nitrogen.

19. The system as claimed in claim 17 or claim 18, wherein the dry gas provided by the first gas source is nitrogen.

20. The system as claimed in any one of claims 17 to 19, further comprising a second gas source for providing a flow of a second gas through the chamber, whereby the flow facilitates release of the sample from the sorbent material.

21. The system of claim 20, wherein the second gas is nitrogen.

22. The system as claimed in any one of claims 17 to 21, wherein the system further comprises
a humidity sensor for sensing the humidity of the dried, released sample and
a controller to monitor and control the flow of the dry gas in response to the sensed humidity.

23. The system as claimed in any one of claims 17 to 22, wherein the system further comprises
a thermal control module to monitor and control the temperature of the mixed dried gas before capture by the recapture device.

24. The system as claimed in any one of claims 17 to 23, wherein the recapture device comprises one or more sorbent tubes.
